(19) United States
(12) Patent Application Publication
(10) Pub. No.: US 2018/0322943 A1
(43) Pub. Date: Nov. 8, 2018

(54) Patient Outcome Tracking Platform

(71) Applicant: Avent, Inc., Alpharetta, GA (US)

(72) Inventors: Dustin Johnson, Johns Creek, GA (US); Marc Comtois, Irvine, CA (US); Kunal M. Amin, La Mirada, CA (US); Kenneth C. Hsu, Tustin, CA (US); Arne Jørgen Madsen, Newport Beach, CA (US); Bao-Tram N. Pham, Irvine, CA (US)

(21) Appl. No.: 15/773,258

(22) PCT Filed: Nov. 10, 2016
(86) PCT No.: PCT/US2016/061262

§ 371 Date: May 3, 2018

Related U.S. Application Data
(60) Provisional application No. 62/254,479, filed on Nov. 12, 2015.

(51) Int. Cl. G16H 10/60; G16H 50/70

(52) U.S. Cl. CPC G16H 10/60; G16H 20/10; G16H 50/70

(57) ABSTRACT

Methods and systems for managing patient health data are provided. A patient data tracking system is provided that aggregates patient data to create one or more patient data sets. The system analyzes and/or sorts the patient data set(s) to selectively provide information to different entities. The patient data may be secured such that the patient data set(s) are stored and/or disseminated in compliance with one or more applicable governmental or organizational regulations. As one example, the system can provide targeted information to a physician and healthcare organization that can help the physician and healthcare organization direct a patient's pain management therapy, develop treatment protocols for a patient population, or the like. The platform may use one or more dashboards to disseminate targeted information to the different entities. The dashboards may present information regarding an individual patient or one or more patient populations, e.g., aggregate patient population data or trends.

### CROSS-REFERENCE TO RELATED APPLICATIONS

The present application claims priority to U.S. Provisional Application Ser. No. 62/254,479, filed on Nov. 12, 2015, which is incorporated herein in its entirety by reference thereto.

### FIELD OF THE INVENTION

The subject matter of the present disclosure relates generally to methods and systems for managing patient health data. More particularly, the present subject matter is directed to methods and systems for collecting, analyzing, storing, and disseminating patient health data to improve patient outcomes.

### BACKGROUND

Healthcare providers and organizations, as well as patients, continuously strive to improve patient outcomes and quality of care. Recently, healthcare providers and organizations have shifted more procedures from inpatient to outpatient settings, such as ambulatory surgery centers (“ASCs”). However, several challenges remain that have prevented shifting an increased number of procedures to outpatient settings. As one example, regional anesthesia has penetrated only a limited number of procedures, at least in part because no effective system exists for effectively monitoring and managing the recovery of patients after outpatient procedures. Thus, healthcare providers and organizations, as well as payors, are reluctant to widely adopt regional anesthesia in outpatient procedures, as there is a lack of data to support that patients are well-managed during the recovery process. Moreover, without a direct tie-in to patient therapies, organizations are reluctant to enter the patient discharge management market, such that the market for managing patient care following outpatient discharge remains underserved.

In addition, particularly after the passage of the Affordable Care Act (“ACA”), payors or payor organizations have sought to improve the efficiency of payments and to appropriately incentivize physicians or clinicians. For example, in the wake of the passage of the ACA, physician and clinician compensation more commonly is being tied directly to the quality of care the physician or clinician provides.

Further, patients generally are willing to share health data. Additionally, sensors, e.g., on medical instruments, as well as wearable devices for detecting biofeedback and the like, are becoming ubiquitous. However, the usefulness in healthcare of patient-generated data and data from sensors and wearables remains unproven. Moreover, while some electronic medical data software exists, such software is primarily focused on utilizing healthcare provider-generated data and replacing paper or hardcopy patient records systems with electronic medical records (“EMR”) systems and/or developing patient administration systems (“PAS”). However, a patient-driven system used with therapies and/or devices could better direct patient outcomes, e.g., patient outcomes related to post-surgical pain management, enteral feeding, respiratory airway management, and surgical infection prevention.

Consequently, there is a need for one or more methods and systems for tracking patient outcomes. In particular, a method and system for administering patient health data would be beneficial. Additionally, a method and system for analyzing, storing, and disseminating patient health data to improve patient outcomes would be useful.

### SUMMARY

The present invention provides methods and systems for managing patient health data. In particular, the present invention provides a patient health platform that collects patient-generated data, e.g., from patients, physicians, device sensors, and the like, to create one or more patient data sets. The platform analyzes and/or sorts the patient data set(s) such that the platform may selectively provide targeted or specific information to physicians, patients, healthcare organizations, medical and other device manufacturers, and/or payors. Sorting the patient data set(s) may include securing the patient data such that the platform includes features for storing and disseminating the patient data set(s) in compliance with one or more applicable governmental or organizational regulations. As one example, the platform can provide targeted information to a physician and healthcare organization that can help the physician and healthcare organization direct a patient's pain management therapy, develop treatment protocols for a patient population, or the like. The platform may use one or more dashboards to disseminate targeted information to physicians, patients, healthcare organizations, medical or other device manufacturers, payors, and/or other appropriate recipients. The dashboards may present information regarding an individual patient or one or more patient populations, e.g., aggregate data or trends regarding a patient population. Additional aspects and advantages of the invention will be set forth in part in the following description, may be apparent from the description, or may be learned through practice of the invention.

In one aspect, the present subject matter is directed to a system for tracking patient data. The system comprises a data device for inputting patient data; a data administration tool for aggregating and trending patient data of a patient population; and a dashboard for disseminating one or more patient population trends. In some embodiments, the data administration tool comprises an analysis module that generates the one or more patient population trends. Further, the one or more patient population trends may be devoid of personal identifiers of individual patients. In some embodiments, the data device is a patient data device, and in particular embodiments, a patient inputs patient data into a web-based survey accessed through the patient data device. The system also may comprise a plurality of data devices and a plurality of dashboards.

In another aspect, the present subject matter is directed to a method for tracking patient outcomes. The method comprises receiving patient data through patient inputs to a data collection protocol; aggregating the patient data of a patient population to form an aggregated population data set; controlling access to the patient data; and disseminating a summary of the aggregated population data set. In some embodiments, disseminating the summary of the aggregated population data set comprises disseminating a comparison of two or more therapies. In other embodiments, disseminating the summary of the aggregated population data set comprises disseminating a patient outcome trend of the patient population. The summary may be disseminated to one or more dashboards, and in particular embodiments, the method includes disseminating a first summary to a first dashboard and disseminating a second summary to a second dashboard. The method also may comprise securing the patient data against tampering or unauthorized access. In some embodiments, the method further comprises identifying a patient population to receive access to the data collection protocol; and providing the patient population access to the data collection protocol.

In yet another aspect, the present subject matter is directed to a method for administering patient data. The method comprises establishing a survey protocol; providing a patient access to the survey protocol; collecting patient data inputs through the survey protocol; aggregating patient data inputs collected from a plurality of patients; and disseminating a summary of the aggregated patient data inputs. The survey protocol may be a dynamic survey protocol, and in some embodiments, the dynamic survey protocol configures a current survey presented to a patient based on a previous survey presented to the patient. In other embodiments of the method, the method also comprises determining a patient procedure for a patient population; configuring, based on the patient procedure, the survey protocol for collecting patient inputs; presenting the patient population with prompts; and receiving patient data inputs based on the prompts. In still other embodiments, the method further comprises establishing a first data set to disseminate to a first entity; establishing a second data set to disseminate to a second entity; disseminating the first data set to the first entity; and disseminating the second data set to the second entity. Disseminating the summary of the aggregated patient data inputs may include disseminating a trend of the aggregated patient data.

In still another aspect, the present subject matter is directed to a patient tracking platform that is able to gather patient-generated data, as well as integrate information collected from sensors embedded onto or into patients or devices. Preferably, the platform is a digital platform. The platform may perform data analytics, e.g., predictive analytics, and derive outcome trends, protocol enhancements, and information dashboards for various parties, such as, e.g., patients, physicians, healthcare organizations, and/or payors. The platform may include one or more methods or systems for collecting, analyzing, sorting, storing, and/or disseminating patient data. For example, the platform may collect data inputs from one or more personal computing devices, smartphones, or the like, and the platform may include analytics utilizing the data inputs to derive patient outcomes and/or to analyze usage data for developing a post-operative pain score, specific therapies, or the like. Further, the analytics may develop a specific therapy such as pain management, enteral feeding, or respiratory health, and/or may take into account the patient's use of a specific device, e.g., pain pump, enteral feeding, or respiratory devices. As a specific example, the method and/or system may provide trending of post-operative pain management, which can influence treatment protocols. Additionally, the platform may comprise a method and/or system for disseminating patient data to one or more entities, such as the patient, physician(s), healthcare organization(s), medical device manufacturer(s), and/or payor(s). In one embodiment, the platform may be a system that presents a customized dashboard to each entity, where access to the patient data is controlled for each entity. That is, different pieces of the patient data may be accessible to the different entities or different levels of access may be granted to the different entities. For example, the patient and physician(s) may be able to access all patient data but the medical device manufacturer(s) may be able to access only performance data of one or more medical devices, which is devoid of any personal data of the patient or any data unrelated to the performance of the device(s). Thus, the platform may include a method and/or system for selectively disseminating patient data, such that different entities have access to different levels or types of patient data.

In one embodiment, the platform comprises a method and/or system, such as a mobile application (i.e., a mobile app), for collecting patient-generated data. In another embodiment, the system comprises a web-based data collection protocol for collecting patient-generated data. Other telecommunications systems, devices, or interfaces also may be used to collect patient-generated data. The platform also may comprise an interface for collecting data derived from sensors connected to patient devices, e.g., an infusion pump or the like, or worn by patients, e.g., a wearable heart rate monitor or the like. As appropriate, the platform may include a method and/or system for integrating the data from the patient devices and/or sensors worn by patients with the patient-generated data. In other embodiments, the platform also may comprise a method and/or system for integrating medical procedure information as well as data from patients' electronic medical records (“EMR”) and/or other systems, e.g., from an existing EMR or PAS system, with the patient-generated, patient device, and/or wearable sensor data.

In still other embodiments, the platform secures the patient data in a manner that complies with applicable governmental or organizational regulations. For example, the platform may comply with the requirements of the Health Insurance Portability and Accountability Act (“HIPAA”) such that the platform is HIPAA-compliant. It should be understood that the patient health platform, including one or more methods and/or systems for collecting, analyzing, storing, and disseminating patient health data, may be further configured with any of the additional features as described herein.

These and other features, aspects, and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1A provides a diagram view of a portion of a representative tracking system for a patient tracking system platform in accordance with an exemplary embodiment of the present subject matter.

FIG. 1B provides a diagram view of another portion of the representative tracking system of FIG. 1B.

FIG. 2 provides a chart illustrating a method for tracking patient outcomes according to an exemplary embodiment of the present subject matter.

FIG. 3 provides a chart illustrating a method for tracking patient outcomes according to another exemplary embodiment of the present subject matter.

FIG. 4 provides a chart illustrating a method for administering patient data according to an exemplary embodiment of the present subject matter.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

In general, the present disclosure is directed to methods and systems for tracking patient outcomes, particularly for the development of patient therapies, the management and improvement of medical devices or instruments, and/or to allow payor organizations to tailor physician compensation to patient outcomes or outcome trends. Preferably, the methods and systems are computer implemented, e.g., using one or more Internet-based interfaces. For sake of example only, the following discussion relates to embodiments of the invention drawn to healthcare-related patient outcome tracking platforms. It should be appreciated, however, that the system and method are just as applicable to any manner of tracking platforms. Embodiments of the methods and system disclosed herein may be executed by one or more suitable networked systems, such as, e.g., personal computers, handheld devices, medical devices, databases, and the like. Such system(s) may comprise one or more computing devices adapted to perform one or more embodiments of the methods disclosed herein. Such systems and computing devices may access one or more computer-readable media that embody computer-readable instructions which, when executed by at least one computer, cause the computer(s) to implement one or more embodiments of the methods of the present subject matter. Additionally or alternatively, the computing device(s) may comprise circuitry that renders the device(s) operative to implement one or more of the methods of the present subject matter. Further, components of the presently-disclosed technology may be implemented using one or more computer-readable media. Any suitable computer-readable medium or media may be used to implement or practice the presently-disclosed subject matter, including, but not limited to, diskettes, drives, and other magnetic-based storage media, optical storage media, including disks (including CD-ROMS, DVD-ROMS, and variants thereof), flash, RAM, ROM, and other memory devices, and the like. In addition, to the extent one or more computer-implemented programs are used, the present subject matter is not limited to any particular programming language. It should be understood that a variety of programming languages may be used to implement the present subject matter as described herein, and any references to specific languages are provided by way of illustrative example only.

The present disclosure also makes reference to the transmission of communicated data over one or more communications networks. It should be appreciated that network communications can comprise sending and/or receiving information over one or more networks of various forms. For example, a network can comprise a dial-in network, a local area network (“LAN”), a wide area network (“WAN”), a public switched telephone network (“PSTN”), the Internet, an intranet, or other type(s) of networks. A network may comprise any number and/or combination of hard-wired, wireless, or other communication links.

Moreover, the particular the naming of the components, capitalization of terms, the attributes, data structures, or any other programming or structural aspect is not mandatory or significant, and the mechanisms that implement the invention or its features may have different names, formats, or protocols. Moreover, the systems may comprise and the methods may be implemented via a combination of hardware and software, as described, or entirely in hardware elements. Also, the particular division of functionality between the various components described herein is merely exemplary and not mandatory; functions performed by a single component may instead be performed by multiple components, and functions performed by multiple components may instead performed by a single component.

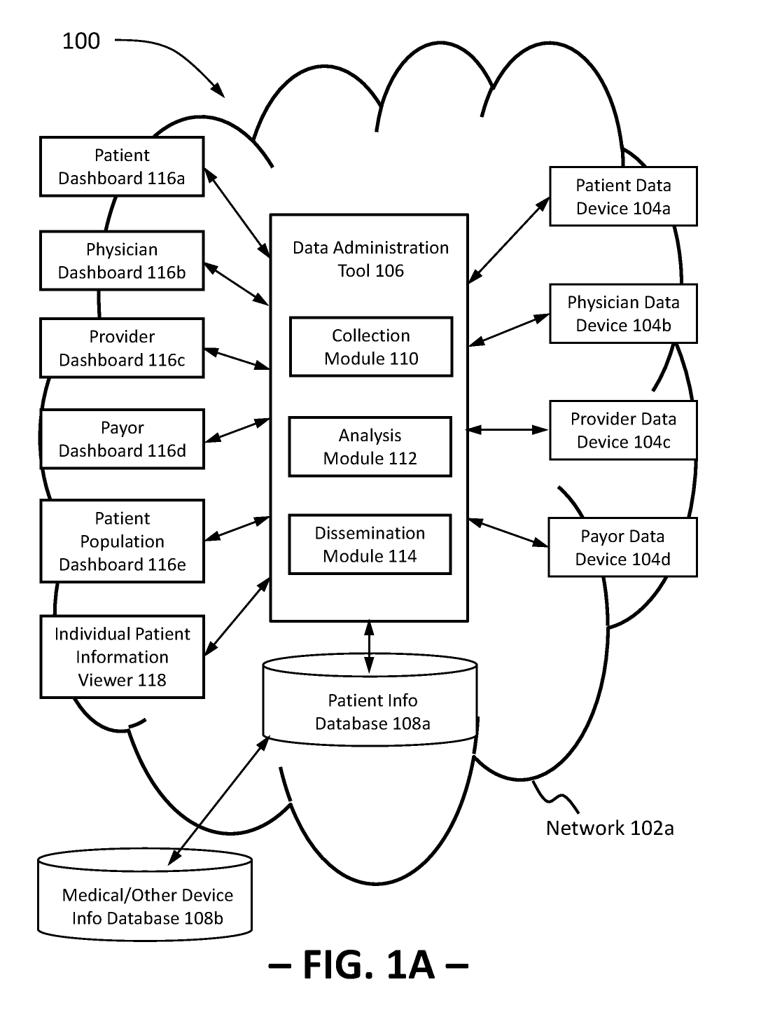

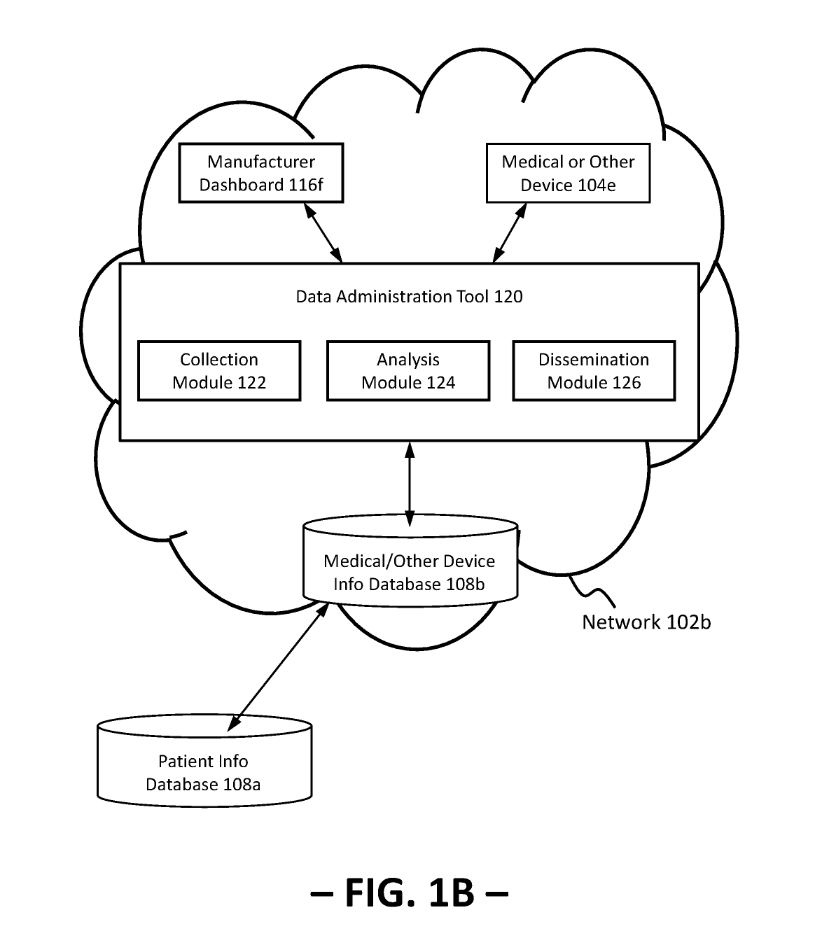

FIG. 1 provides a diagram view of a representative tracking system 100 that may be used to practice aspects of the patient tracking system platform in accordance with an exemplary embodiment of the present subject matter. Tracking system 100 includes a network 102 for sending and/or receiving information or data as previously described. A data device 104 connected through network 102 to a data administration tool 106 may provide patient data to the data administration tool 106 for use in tracking a patient's outcomes. In some embodiments, the tracking system 100 may include a plurality of data devices 104. For example, as shown in FIG. 1, a patient may use a patient data device 104a to provide patient-generated data. As further shown in FIG. 1, a physician may provide physician-generated patient data through a physician data device 104b; a healthcare provider, such as a hospital or other healthcare organization, may provide provider-generated patient data through a provider data device 104c; and a payor organization, such as an insurance company, may provide payor-generated patient data through a payor data device 104d. Additionally or alternatively, each of a patient, a physician, a provider, and a payor may receive patient data through the respective data device 104a, 104b, 104c, 104d. Patient data device 104a, physician data device 104b, provider data device 104c, and payor data device 104d may comprise a personal computing device, such as portable or mobile telecommunications devices, e.g., with Internet functionality. As examples, data devices 104a, 104b, 104c, 104d may be desktop computers, tablet computers, smartphones, or any other suitable personal computing devices. Moreover, one or more medical or other devices or instruments 104e also may be connected to data administration tool 106, or a separate data administration tool 120, to provide patient data for use in, e.g., tracking patient outcomes, developing treatment protocols, refining the configuration or functionality of the medical or other device, etc. Tracking system 100 also may comprise other data devices 104.

The data devices 104 are configured to execute one or more computer programs, such as an Internet browser program, to allow users to interact with the data administration tool 106, a data collection protocol 105, and/or dashboards 116 described below, and preferably include a visual display such as a monitor or screen. Alternatively, the visual display may be incorporated into a web-browser configured to display multimedia content. For instance, a user, such as a patient or a physician, may provide data to data administration tool 106 remotely via an Internet web-browser on data devices 104. Further, the medical or other devices 104e may or may not include a display but may provide data that is incorporated into one or more dashboards or other information summaries provided to a display of another data device 104.

The patient data may include patient-generated data, physician-generated data, provider-generated data, payor-generated data, medical device-generated data, and the like. Patient-generated data comprises patient inputs as to a patient's experiences or the patient's subjective feedback concerning an event, a device, or other similar patient inputs. In some embodiments, patient data device 104a may comprise a browser through which a patient accesses a web-based data collection protocol 105 for gathering or collecting patient-generated data. The web-based data collection protocol 105 may be, e.g., a web-based survey protocol having a log portion, a questionnaire portion, and the like. For instance, a portion of the survey may be a log where the patient can, e.g., rate his or her pain or relative pain level once a day or throughout the day, rate the patient's perceived or subjective recovery level, indicate the patient's activity level, or the like. Another portion of the survey may be configured similar to a questionnaire, presenting the patient with questions or prompts and allowing the patient to select a pre-generated answer or input an answer. That is, the survey may present the patient with answers to choose from, may allow free-form answers, or a combination of both. Further, the survey may be tailored to the patient's specific procedure or a device the patient is using. As an example, if the patient is using a post-operative infusion pump for regional anesthetic, the survey can present the patient questions or prompts specific to infusion pumps and pain management. Additionally, the survey protocol may be a dynamic survey protocol that, e.g., presents questions or prompts to the patient based on past responses, past non-responses, or previously collected data. For example, based on an answer by the patient to a previous survey, the survey may ask a follow-up question or omit a question related to the previous answer. That is, the survey may utilize the patient's previous answers or failures to answer to determine current survey questions or prompts. The survey may have other functionalities or configurations as well.

In other embodiments, the data collection protocol 105 may be a mobile application, i.e., an app, designed to capture patient inputs, and the app may be used in place of or in addition to the web-based survey to gather patient data. For instance, a patient may download the app onto his or her smartphone before or after a medical procedure to track the patient's recovery, or the patient may download the app as part of tracking the patient's health in general. The app may be configured similarly to the above-described survey, e.g., with a log portion, a questionnaire portion, and the like. In still other embodiments, the data collection protocol 105 may receive patient inputs via email, SMS text messages, or other means of communicating information. The data collection protocol 105 may have other configurations as well.

Moreover, although described above with respect to one patient, it should be appreciated that the tracking system 100 may receive patient-generated data from any number of patients, e.g., through any number and variety of patient data devices 104a. As described in greater detail herein, the tracking system 100 may compile a data set for each individual patient who provides information to the tracking system 100, as well as aggregate each patient data set received by the tracking system 100. The tracking system 100 may analyze the aggregate patient data and, e.g., produce one or more trends for one or more patient populations. For example, from the aggregated patient data, the tracking system 100 may produce a trend for a population of patients utilizing a certain medical device or a population of patients recovering from a certain medical procedure.

Physician-generated data comprises physician inputs as to the physician's observations, test results, or similar data relating to the physician's patients. It will be appreciated that “physician” as used herein may refer to any type of caregiver who provides medical care to a patient, whether a medical doctor, a nurse practitioner, a registered nurse, a clinician, or other caregiver. In some embodiments, a physician data device 104b may comprise or be in communication with an electronic medical record (“EMR”) system such that the physician inputs are captured as part of a patient's electronic medical records. In other embodiments, the physician may use a data collection protocol 105, such as one or more mobile applications (i.e., apps), web-based surveys, or the like, to input physician-generated data. Further, although described herein with respect to one physician, it should be understood that any number of physicians, clinicians, or caregivers may be involved in a patient's care and each physician, clinician, or caregiver may generate data specific to the patient that generally is described herein as physician-generated data. Moreover, as described above, the tracking system 100 may receive patient-generated data for any number of patients, and similarly, the tracking system 100 may receive physician-generated data for any number of patients from any number of physicians, clinicians, or caregivers.

Provider-generated data may comprise data related to a patient's interaction with a healthcare organization. For example, provider-generated data may include details related to a patient's hospital stay, a patient's interactions with provider personnel upon check-in for an appointment, or the like. In some embodiments, provider inputs through, e.g., a provider data device 104a, may be capture as part of a patient administration system (“PAS”). Provider-generated data may comprise other information as well.

Payor-generated data may comprise data related to a patient's treatment and outcomes or progress. For instance, payor-generated data may include what treatments are available for a given diagnosis, the relative cost of treatment options, the cost of the patient's treatment over time, the patient's outcome to date versus other patient's outcomes using the same treatment for the same amount of time, etc. Other data also may be payor-provided.

Medical device-generated data comprises data from one or more medical devices or instruments 104e used by a patient. Medical device-generated data also may include data from one or more wearable or non-wearable devices, e.g., for detecting the patient's vitals, biofeedback, biomarkers, and/or the patient's activity, such as the patient's heartrate, number of footsteps taken, number of times the patient has stretched a limb (i.e., stretching reps), etc. For example, each medical or other device may have one or more sensors that output data regarding, e.g., the patient's body functions, the device's performance, device usage, or the like. Moreover, although referred to herein together with medical devices or instruments under the term “medical devices,” patient wearable and non-wearable devices may or may not be medically-related, i.e., the devices may or may not be prescribed or monitored by the physician or generally described as a medical device or instrument. Thus, medical device-generated data as used herein generally refers to the inputs, outputs, or data from one or more sensors of medical devices and/or other devices utilized by the patient, such as the wearable and non-wearable devices described above.

As a specific example, a medical device 104e may be an infusion pump that delivers medication to a patient intended to alleviate the patient's pain and that has at least one sensor for sensing the amount of infusion fluid dispensed via the pump over a period of time. As another example, the medical device may include one or more cameras that, e.g., transmit images from the point of view of the camera. Additionally or alternatively, the camera may incorporate GPS tracking and/or motion tracking capabilities, as well as one or more environmental sensors. In one embodiment, a patient's motion may be tracked using one or more accelerometers. Of course, GPS tracking capability, motion tracking capability, and environmental sensors may be incorporated into or onto other medical devices or independently of a medical device or camera. For example, motion tracking capability, such as one or more accelerometers, can be integrated into a wearable device, e.g., a vest, harness, wristband, or the like, and can provide feedback as to a patient's steadiness, limb paresthesia, or other conditions.

As will be readily understood, using data devices 104, the data administration tool 106 may be provided with real-time feedback as to the patient's status and with real-time data regarding the patient's outcomes. For example, the medical device-generated data may be continuously delivered to the data administration tool 106 substantially in real-time. Further, using patient data device 104a, the patient can input data at the time of an event or immediately thereafter. Thus, the collection of patient data may occur substantially in real-time. It will also be appreciated, as further described below, that the dissemination of the patient data also may occur substantially in real-time. Additionally or alternatively, the data administration tool 106 may be integrated with one or more EMR and/or PAS systems, e.g., for sharing of patient data between the data administration tool 106 and the EMR and/or PAS systems. As such, it will be understood that the receipt and/or dissemination may occur substantially in real-time, on a specified schedule, or essentially at any time. For instance, the data administration tool 106 may comprise instructions for the receipt and/or dissemination of data according to a schedule, upon satisfaction of one or more conditions, or the like.

The data administration tool 106 is configured to respond to inputs through data devices 104 to provide tracking and management of the patient's outcomes, e.g., the patient's pain management following an outpatient procedure. The data administration tool 106 comprises computer logic utilized to provide the specified functionalities of data administration tool 106. More particularly, as shown in FIG. 1A, the data administration tool 106 comprises a number of processing modules. It will be appreciated that the term “module” refers to computer logic utilized to provide the specified functionality of the module. Thus, a module can be implemented in hardware, firmware, and/or software controlling a general purpose processor. In one embodiment, the modules are program code files stored on the storage device, loaded into memory, and executed by a processor. Alternatively, the modules can be program code files provided from computer program products, e.g., computer executable instructions, which are stored in a tangible computer-readable storage medium such as RAM hard disk or optical or magnetic media. Also, it will be appreciated that embodiments of data administration tool 106 can have different or other modules than the ones described herein, with the described functionalities distributed amongst the modules in a different manner.

Further, the data administration tool 106 may be hosted on a server that is cloud-based, co-located at a provider site, or located at another appropriate site. Additionally, an administrator may have access to the data administration tool 106 to refine the logic or other aspects of the data administration tool 106. It will be appreciated that the administrator may create the logic utilized by data administration tool 106, including the data collection protocol 105 and the various modules of tool 106. The administrator may perform other duties as well, such as managing users, e.g., patients, physicians, etc., of the data administration tool 106.

Data administration tool 106 may respond to the input of patient data by storing the data in one or more databases 108, such as patient information database 108a, communicatively connected to data administration tool 106. The patient information database 108a provides a data repository for the storage and correlation of information gathered from data devices 104. That is, patient information database 108a aggregates, e.g., the patient-generated data, physician-generated data, and other data forming one or more patient data sets as described above. As such, the information stored within the patient information database 108a may be information relating, e.g., to patients' subjective inputs, medical device readings, and test results input by the patients' physician(s). The patient information database 108a may provide information specific or personal to a patient or information regarding a patient population, e.g., a data set devoid of personal identifiers but identifying a trend among a population of patients.

Additionally, as shown in FIGS. 1A and 1B, the data administration tool 106 may be provided access to other databases 108, such as a medical or other device information database 108b, to provide data administration tool 106 with information needed to track and/or manage patient outcomes, develop treatment protocols, or the like. For instance, referring to FIG. 1B, the medical/other device information database 108b may store data from the medical or other devices 104e described above, and database 108b may be linked to database 108a such that database 108b can provide data to data administration tool 106. The data administration tool 106 may be provided access to other databases 108 as well. For example, a clinical database (not shown) may provide information about different therapies that may be used to manage patient recovery. In general, clinical database provides information that is non-specific or non-personal to a patient, i.e., common information about therapies, devices, and the like without any reference to patient data. In some embodiments, general information databases such as the clinical database may be omitted, and other databases may or may not be provided. It should also be understood that separate databases 108 may be provided for different sets of patient data, e.g., one database 108 may be provided for patient data of one provider, another database 108 may be provided for patient data of another provided, etc., and the data administration tool 106 may access each database 108.

Referring to FIG. 1A, the data administration tool 106 is configured to collect the patient data, e.g., for storage in patient information database 108a, to analyze the patient data, and to disseminate patient data; the data administration tool 106 may have other functionalities as well. More specifically, the data administration tool 106 comprises a collection module 110 for collecting patient data from data devices 104. Collecting the patient data may comprise connecting to one or more data devices 104, e.g., through network 102a as described herein. One or more pieces of patient data may be sent to or received by an analysis module 112 for analysis, which may comprise sorting the data in preparation for analyzing or disseminating the data. In particular, the analysis module 112 includes one or more algorithms to analyze patient behaviors and attitudes, medical device usage, medication usage, and the like to, e.g., perform predictive analytics to trend a patient's outcomes, a patient population's outcomes, or other information regarding a specific patient or a group of patients. For example, analysis module 112, using inputs from a patient data device 104, a physician data device 104b, and/or a medical/other device(s) 104e, may develop a post-operative pain score for a patient, which can be used to develop or modify a treatment protocol for the patient. Further, analysis module 112 may use the patient data to develop other specific therapies, such as, e.g., pain management using a pain pump, enteral feeding using an enteral feeding device, or respiratory health using a respiratory device. As an example, the method and/or system may utilize predictive analytics to provide trending of post-operative pain management based on a patient's use of a pain pump. The analysis module 112 may comprise a rules engine, transformation logic, or the like for performing analysis functions.

The patient data or the results of the analysis of the patient data may be selectively disseminated or distributed via dissemination module 114. At least a portion of the patient data may be available to one or more entities, such as the patient, physician(s), provider(s) or healthcare organization(s), device manufacturer(s), and/or payor(s). In one embodiment, dissemination module 114 is configured to present a customized dashboard 116 to each entity, such that the tracking system 100 comprises a plurality of dashboards 116. For instance, the dissemination module 114 of data administration tool 106 may comprise parameters for each dashboard 116 such that each dashboard 116 displays a visual representation of different patient data or different subsets of patient data. More particularly, as further described herein, access to the patient data may be restricted such that different entities have different levels of access to the patient data. The dashboard parameters may ensure that each dashboard 116 displays only patient data accessible by the entity to which the particular dashboard 116 is provided. Further, the dissemination module 114 may comprise logic or the like for transforming the patient data into a visual representation or summary of the data, such as a graph, chart, or other pictorial representation of the patient data.

In the exemplary embodiment illustrated in FIG. 1A, dissemination module 114 distributes data to a patient dashboard 116a, a physician dashboard 116b, a provider dashboard 116c, a payor dashboard 116d, and a patient population dashboard 116e. As described above, the dashboards 116 may be graphical or other visual representations of patient data, such as charts, graphs, or the like, that may be summaries of the patient data or a portion of the patient data. For example, each patient that interacts with tracking system 100 may have access to a patient dashboard 116a for receiving a summary of the patient's data. The patient dashboard 116a for a specific patient may provide a graphical summary of the patient's survey responses, a trend of the patient's progress toward a health goal, and/or a comparison of the patient's progress to the average progress of a group of patients toward the same health goal. The other dashboards 116 similarly may provide summary information to the targeted entity; for instance, the physician dashboard 116b may provide a physician a summary of an individual patient's data, a summary of the data of a subset of the physician's patients, and/or other data summaries. As further described below, for some entities that have access to one or more dashboards 116, the data administration tool 106 may secure the patient data such that the entity may view only non-identifiable patient data or data summaries, e.g., a trend or summary for one or more patient populations or a trend or summary for an individual patient that does not contain any personal identifiers for the individual patient.

The dissemination module 114 also may distribute data to a viewer 118 for viewing individual patient information. That is, the individual patient information viewer 118 may provide raw patient data to an entity authorized to access such data. For example, a physician may be given access to the raw patient data of the physician's patients. The physician may access the physician's patients' data, e.g., by logging in to the individual patient information viewer 118, which grants the physician access to only the physician's patients' data.

As shown in FIG. 1B, the medical/other device data may be analyzed through a data administration tool 120 that is similar to the data administration tool 106. More particularly, the data administration tool 120 comprises computer logic that is utilized to provide the specified functionalities of data administration tool 120. As shown in FIG. 1B, the data administration tool 120 comprises a collection module 122, an analysis module 124, and a dissemination module 126. Also, it will be appreciated that embodiments of data administration tool 120 can have different or other modules than the ones described herein, with the described functionalities distributed amongst the modules in a different manner. Further, the data administration tool 120 may be hosted on a server that is cloud-based, co-located at a provider site, or located at another appropriate site.

Data administration tool 120 receives data from one or more medical or other devices 104e. The data administration tool 120 may respond to the input of such patient data by storing the data in one or more databases 108, such as medical or other device information database 108b described above, which is communicatively connected to data administration tool 120. The medical/other device information database 108b provides a data repository for the storage and correlation of information gathered from medical or other devices 104e. That is, database 108b aggregates data from one or more medical or non-medical devices 104e, such as medical or other device readings, performance, resets, faults, or the like. Further, as shown in FIGS. 1A and 1B, database 108b may be communicatively connected to patient information database 108a to provide data from devices 104e for use in one or more patient data sets. The medical/other device information database 108b may provide information specific or personal to a patient or information regarding a patient population, e.g., a data set regarding a specific device used by a plurality of patients.

Similar to data administration tool 106, the data administration tool 120 illustrated in FIG. 1B is configured to collect medical or other device data, e.g., for storage in database 108b, analyze the medical or other device data, and disseminate such data; the data administration tool 120 may have other functionalities as well. More specifically, the collection module 122 collects patient data from medical or other devices 104e. One or more pieces of such data may be sent to or received by the analysis module 124 for analysis, which may comprise sorting the data in preparation for analyzing or disseminating the data. In particular, the analysis module 124 includes one or more algorithms to analyze, e.g., medical or other device usage, performance, and the like to trend a device's error rate, usefulness in patient treatment, or other information regarding one or more devices. The medical/other device data or the results of the analysis of such data may be selectively disseminated or distributed via dissemination module 126. At least a portion of the medical/other device data may be available to one or more entities, such as the patient, physician(s), provider(s) or healthcare organization(s), medical device manufacturer(s), and/or payor(s). In one embodiment, dissemination module 126 is configured to present a customized dashboard 116f to each device manufacturer. The dashboard 116f may be a graphical or other visual representation of medical/other device data, such as charts, graphs, or the like, that may be summaries of a portion of the device data. The medical/other device data, or summaries of such data, also may be presented through one or more of the dashboards 116 or the patient information viewer 118 illustrated in FIG. 1A.

Preferably, access to the patient data is controlled for each entity. That is, different pieces of the patient data may be accessible to the different entities or different levels of access may be granted to the different entities. As one example, a patient and the patient's physician(s) may be able to access all data related to that patient, but the medical device manufacturer(s) may be able to access only performance data from medical/other device(s) or instrument(s) 104e, which is devoid of any personal data of the patient or any data unrelated to the performance of the device(s). Accordingly, disseminating the patient data may include sorting the data such that the appropriate portion of the data is distributed or made accessible to the appropriate entity.

In some embodiments, patient dashboard 116a is viewable or accessible using patient data device 104a. For example, patient dashboard 116a may be a component of the survey or mobile app that also accepts data inputs by the patient as described above. Alternatively or additionally, the patient may access or view patient dashboard 116a separately from the survey or app, e.g., using the browser of patient data device 104a. Similarly, physician dashboard 116b may be viewable or accessible using physician data device 104b. In other embodiments, patient dashboard 116a and physician dashboard 116b may be standalone components separate from data devices 104a, 104b. Of course, provider dashboard 116c, payor dashboard 116d, and manufacturer dashboard 116f may be viewable or accessible via provider, payor, and manufacturer devices, respectively, that are similar to patient and physician data devices 104a, 104b. In other embodiments, provider dashboard 116c, payor dashboard 116d, and manufacturer dashboard 116f may be standalone components or viewable or accessible from other devices, and such devices may include an interface for the receipt of provider-generated data, payor-generated data, medical/other device-generated data, and manufacturer-generated data. The provider-generated data, payor-generated data, medical/other device-generated data, and manufacturer-generated data may then be viewable or accessible by the patient, the physician, and/or other appropriate entities. Also, the patient population dashboard 116e and individual patient information viewer 118 may be viewable or accessible through one or more data devices 104 or standalone components similar to the dashboards 116a, 116b, 116c, 116d, 116f.

As further depicted in FIG. 1A, data devices 104, data administration tool 106, databases 108, and dashboards 116 are connected and/or multiplexed to network 102a, e.g., via direct network or other suitable links. Similarly, as depicted in FIG. 1B, the medical or other devices 104e, data administration tool 120, database 108b, and manufacturer dashboard 116f are connected and/or multiplexed to network 102b, e.g., via direct network or other suitable links. Preferably, the links in each network 102a, 102b are secure communications channels that include features for preventing tampering with or unauthorized access to the information transmitted using the channels. For example, the communications channels are physically hardened against tampering or are encrypted to prevent unauthorized access to information transmitted thereon. In an exemplary embodiment, the links are secured in a manner that complies with applicable governmental or organizational requirements. As an example, the Health Insurance Portability and Accountability Act (“HIPAA”) regulates access to patient data, and the links may be secured consistent with such regulations to prevent unauthorized access under HIPAA. Accordingly, in an exemplary embodiment, tracking system 100 is HIPAA-compliant.

Similarly, the data administration tools 106, 120 preferably include features for preventing tampering with or unauthorized access to the patient data and, as such, are secure tools. The data administration tools 106, 120 may include such features for securing the patient data in addition to or separately from the features provided to secure the network links as previously described. In some embodiments, a secure data administration tool is one that complies with applicable governmental or organizational regulations. For example, data administration tools 106, 120 may be configured to comply with the requirements of HIPAA such that each data administration tool 106, 120 is HIPAA-compliant. In other embodiments, the data administration tools may include other features or meet other requirements to prohibit tampering with and unauthorized access of the patient data and, thus, be a secure tool. Similar to the network links and data administration tools 106, 120, the patient information database 108a and medical/other device information database 108b may be secured against tampering and unauthorized access and, for example, may conform to HIPAA or other requirements for securing patient data.

It should be appreciated that, in some embodiments, collection module 110, analysis module 112, and/or dissemination module 114 may be separate from data administration tool 106. That is, modules 110, 112, 114 may be standalone components of system 100 in communication with the other components of system 100, e.g., data devices 104, databases 108, and dashboards 116, via network 102a. Similarly, collection module 122, analysis module 124, and/or dissemination module 126 may be separate from data administration tool 120 such that modules 122, 124, 126 may be standalone components of system 100 in communication with the other components of system 100. System 100 may have other configurations as well.

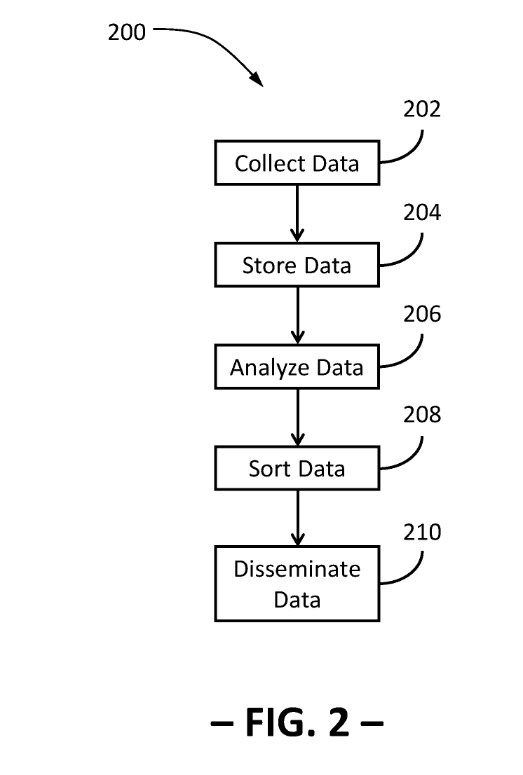

Referring now to FIG. 2, a chart is provided illustrating a method for utilizing tracking system 100, e.g., to track patient outcomes, according to an exemplary embodiment of the present subject matter. As shown, method 200 comprises the steps of collecting patient data and disseminating patient data and also may include the steps of storing, analyzing, and sorting patient data. For example, as described above, patient data may be collected using one or more data devices 104, such as patient data device 104a, physician data device 104b, provider data device 104c, payor data device 104d, and medical/other device 104e. The patient data may then be stored, or it may be analyzed and then stored. Alternatively, the patient data may be sorted, then stored, or stored, then sorted, and then analyzed. In the illustrated exemplary embodiment, method 200 includes step 202 of collecting patient data; step 204 of storing patient data; step 206 of analyzing patient data; step 208 of sorting patient data; and step 210 of disseminating patient data. In other embodiments, the patient data may be stored, sorted, and/or analyzed in any order and any number of times after it is collected and before it is disseminated. In addition, the patient data may be continually collected and disseminated, with any number and order of storing, sorting, and/or analyzing steps included. Thus, FIG. 2 depicts only an exemplary embodiment of method 200, and the steps of method 200 may be reorganized and repeated as necessary to sufficiently track patient data and thereby track patient outcomes.

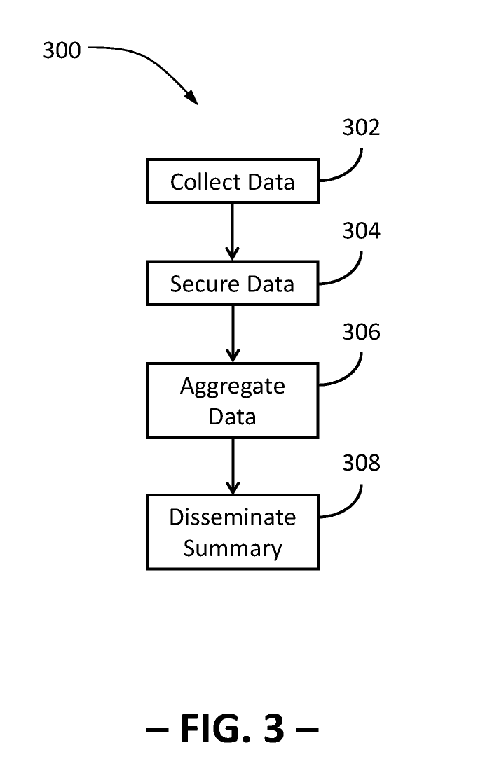

The patient tracking platform may encompass other methods as well. In an exemplary embodiment illustrated in FIG. 3, a method 300 for tracking patient outcomes is provided. The method 300 includes step 302 of collecting patient data of a patient population. The patient population preferably comprises a plurality of patients and, for example, may comprise a plurality of patients who have undergone the same medical procedure or who have a common health goal. The patient data may be inputs received through one or more data devices 104 as described above, and the method for tracking patient outcomes may further comprise inputting the patient data through a plurality of data devices 104. The patient data may be collected by a collection module 110 of a data administration tool 106 as previously described. That is, one or more network connections between the data device(s) 104 and the data administration tool 106 may facilitate the collection of the patient data by the collection module 110. The method 300 for tracking patient outcomes further comprises aggregating the patient data of the patient population to form an aggregated population data set, as shown at 306 in FIG. 3. The patient data may be aggregated by an analysis module 112 of the data administration tool 106 as described above. Moreover, aggregating the patient data may include compiling, organizing, sorting, etc. the patient data for analysis or for dissemination. The method 300 for tracking patient outcomes also includes disseminating a summary of the aggregated population data set, as shown at 308 in FIG. 3. The summary may be a graphical summary or the like disseminated to one or more dashboards 116 as previously described. In one embodiment, disseminating the summary of the aggregated population data set comprises disseminating a first summary to a first dashboard 116 and disseminating a second summary to a second dashboard 116. Each summary may be tailored to a specific entity, e.g., the first summary may contain some personally identifiable patient information while the second summary does not contain any personally identifiable patient information.

In some embodiments, disseminating the summary of the aggregated population data set comprises disseminating a comparison of two or more therapies. For instance, the patient data may include information regarding the progress of the patients in the patient population with respect to a treatment therapy for attaining a health goal. A database 108 in communication with the data administration tool 106 may contain information regarding another patient population's progress with respect to a different therapy for attaining the same health goal. The summary compares the two patient population's progress with respect to the two different therapies. Alternatively, the method 300 may comprise, at step 302, collecting patient data of two or more patient populations, where each patient population is subjected to a different therapy. The method 300 may further comprise, at step 306, aggregating the patient data of the patient populations to form aggregated population data sets and disseminating a summary that compares the two or more therapies.

In another embodiment, disseminating the summary of the aggregated population data set comprises disseminating a patient outcome trend of the patient population. For example, the patient data may include information regarding the outcomes of the patients within the patient population. The patient outcome information may be aggregated into a trend such that the trend provides the patients' outcomes over time.

The method 300 for tracking patient outcomes may further comprise securing the patient data against tampering or unauthorized access, as shown at 304 in FIG. 3. As previously described, the data administration tools 106, 120 and databases 108 that manage the patient data preferably include features for preventing tampering with or unauthorized access to the patient data and, for example, comply with applicable governmental or organizational regulations for securing patient information. In some embodiments, the patient data may be secured in a manner that complies with the requirements of HIPAA. Securing the patient data may include, e.g., assigning different levels of access to different entities. For example, a physician may be provided complete access to patient data of the physician's patients while a medical device manufacturer is provided access to only a portion of the patient data, e.g., a portion of the patient data that does not contain any personally identifiable patient information.

Methods for administering patient data may be provided as well. In an exemplary embodiment, a method for administering patient data includes establishing a survey protocol. The method for administering patient data also may include providing a patient access to the survey protocol and collecting patient data inputs through the survey protocol. In some embodiments, the survey protocol is a web-based survey, but as described above, in other embodiments the survey protocol may be a mobile app downloaded on the patient's smartphone. Further, the survey protocol may be a dynamic survey protocol, which may configure a current survey presented to a patient based on a previous survey presented to the patient, as previously described.

The method for administering patient data also may include aggregating patient data inputs collected from a plurality of patients and disseminating a summary of the aggregated patient data inputs. Disseminating the summary of the aggregated patient data inputs may include disseminating a trend of the aggregated patient data. The trend may, e.g., provide a visual representation of any changes in the aggregated patient data over time.

In some embodiments, the method for administering patient data may further comprise determining a patient procedure for a patient population and configuring, based on the procedure, the survey protocol for collecting patient inputs. The method also may include presenting the patient population with prompts and receiving patient data inputs based on the prompts. The prompts may be questions or statements eliciting one or more responses from the patient as described above with respect to data collection protocol 105. The survey protocol may be presented to each individual patient within the patient population through a patient data device 104a of the individual patient. Further, the patient procedure may be an operation or other medical procedure that each patient of the patient population has undergone. In some embodiments, the patient population may comprise a plurality of patients who have undergone the patient procedure within a certain time frame, e.g., within the past six months or within the past year.

In other embodiments, the method for administering patient data further comprises establishing a first data set to disseminate to a first entity and establishing a second data set to disseminate to a second entity, then disseminating the first data set to the first entity and disseminating the second data set to the second entity. As an example, the first data set may be the patient data of a patient population consisting of a physician's patients, and the second data set may be a portion of the patient data without any personally identifiable information. The first data set may be disseminated to the physician, and the second data set may be disseminated to a medical device manufacturer or a payor organization.

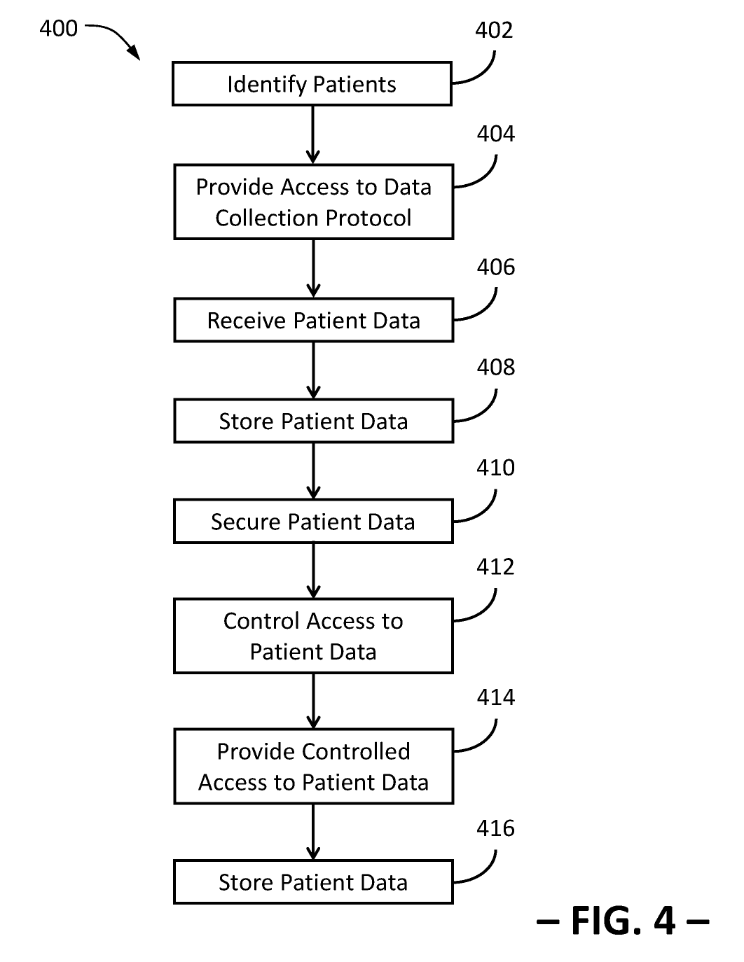

Other methods for administering patient data also may be provided. In one exemplary embodiment illustrated in FIG. 4, a method 400 for administering patient data is provided. The method 400 comprises, at step 402, identifying one or more patients, e.g., an individual patient or a patient population, to receive access to a data collection protocol, such as the data collection protocol 105 previously described. The method 400 further comprises providing the one or more patients access to the data collection protocol and receiving patient data through patient inputs into the data collection protocol, as shown at 404 and 406, respectively, in FIG. 4. Additionally, the method 400 includes steps 408 and 410 of storing and encrypting the patient data (e.g., securing the patient data as previously described), as well as step 412 of controlling access to the patient data, for example, through a data administration tool 106. Storing the patient data may comprise aggregating the patient data to form an aggregated data set. For instance, patient data may be received from a patient population, such that aggregating the patient data includes aggregating the patient data of the patient population to form an aggregated population data set.

The method 400 of administering patient data also includes steps 414 and 416 of providing controlled access to the patient data and disseminating the patient data, e.g., presenting a summary of the aggregated population data set through one or more dashboards 116. In some embodiments, access to the patient data may be controlled by identifying multiple layers of patient data and selectively providing access to one layer, a portion of the layers, or all layers of the patient data, e.g., through a password-protected login system. The layers of patient data may comprise different levels of personal identifiers. For example, a first layer may comprise no personal identifiers, a second layer may comprise some identifying information, a third layer may comprise more personally identifying information than the second layer, and the fourth layer may comprise all personal identifiers of the patient or patients. Different entities, e.g., patients, physicians, providers, payors, and device manufacturers, may be provided different levels of access to the layers of patient data. Further, disseminating the patient data may include disseminating a summary of the patient data by disseminating a comparison of two or more therapies or disseminating a patient outcome trend of the patient population, as described in greater detail above.

The patient tracking platform also may include a method for gathering or collecting patient data. Such method may include, e.g., establishing a procedure a patient has undergone or will undergo; configuring, based on the procedure, a data collection protocol 105, such as an app for patient data device 104a or a web-based survey for display on patient data device 104a; presenting the patient with questions or prompts; and receiving patient input. The platform also may include a method for presenting the questions or prompts to the patient, e.g., comprising selecting appropriate questions or prompts from a database of questions or prompts based on the patient's procedure, the patient's progress, and the intended outcome of the patient. The questions or prompts may be presented to the patient using patient data device 104a. In some embodiments, the data collection protocol is a dynamic data collection protocol, such that the questions or prompts presented to the patient may change between presentations of the data collection protocol to the patient. That is, the patient may access the data collection protocol on separate occasions, and the data collection protocol may present one or more questions or prompts to the patient that are different from questions or prompts previously presented to the patient.

Further, the patient outcome tracking platform also may include a method for extracting portions or subsets of the patient data for dissemination to different entities. Generally, such method may include determining what data is included in the patient data; establishing a first data set to distribute to a first entity; establishing a second data set to distribute to a second entity; distributing the first data set to the first entity; and distributing the second data set to the second entity. Determining what data is included in the patient data may include determining whether and what personal identifiers are present in the patient data. As such, when the patient data is divided into portions to be disseminated, the personal identifiers can be separated out, or access to the personal identifiers can be restricted, such that the personal identifiers are not disseminated to or accessed by inappropriate entities, e.g., device manufacturers. Of course, it will also be understood that the patient data may be divided into other data sets, e.g., a third data set, a fourth data set, etc., and disseminated to other entities, e.g., a third entity, a fourth entity, etc. Additionally, the same data set may be distributed or disseminated to different entities, e.g., the entire set of an individual patient's patient data may be disseminated to both the patient and the patient's physician, but only a portion of the patient's patient data is disseminated to a payor organization and a different portion or data set is distributed to a device manufacturer.

It will be appreciated that the methods and/or systems described herein provide several benefits or advantages. As one example, the integrated patient data, i.e., the collected and compiled patient-generated, physician-generated, medical device-generated, provider-generated, and payor-generated data generally referred to herein as patient data, can provide insight and trending of post-operative pain management or other health goals for an individual patient or one or more patient populations and thereby be used to influence or develop treatment protocols. More particularly, the patient data, e.g., through trending or the like, can be used to develop or modify treatment protocols for an individual patient or a patient population. As another example, the methods and/or systems may facilitate the use of the integrated data to allow physicians to compare the effectiveness of various treatment modalities, e.g., in post-operative pain management. Moreover, device manufacturers can use the integrated data, or a portion or subset thereof, to determine the effectiveness of a medical device, areas for improvement in the device (e.g., potential product design improvements), and the device's contribution to a patient's or a patient population's outcome. Further, the integrated data, or a portion or subset thereof, can be used by payor organizations to adjust compensation to physicians based on patient outcomes or outcome trends.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

1. A system for tracking patient data, the system comprising:
a data device for inputting patient data;
a data administration tool for aggregating and trending patient data of a patient population; and
a dashboard for disseminating one or more patient population trends.

2. The system of claim 1, wherein the data administration tool comprises an analysis module that generates the one or more patient population trends.

3. The system of claim 1, wherein the one or more patient population trends are devoid of personal identifiers of individual patients.

4. The system of claim 1, wherein the data device is a patient data device.

5. The system of claim 4, wherein a patient inputs patient data into a web-based survey accessed through the patient data device.

6. The system of claim 1, wherein the system comprises a plurality of data devices and a plurality of dashboards.

7. A method for tracking patient outcomes, the method comprising:
receiving patient data through patient inputs to a data collection protocol;
aggregating the patient data of a patient population to form an aggregated population data set;
controlling access to the patient data; and
disseminating a summary of the aggregated population data set.

8. The method of claim 7, wherein disseminating the summary of the aggregated population data set comprises disseminating a comparison of two or more therapies.

9. The method of claim 7, wherein disseminating the summary of the aggregated population data set comprises disseminating a patient outcome trend of the patient population.

10. The method of claim 7, wherein disseminating the summary of the aggregated population data set comprises disseminating the summary to one or more dashboards.

11. The method of claim 7, wherein disseminating the summary of the aggregated population data set comprises disseminating a first summary to a first dashboard and disseminating a second summary to a second dashboard.

12. The method of claim 7, further comprising securing the patient data against tampering or unauthorized access.

13. The method of claim 7, further comprising:
identifying a patient population to receive access to the data collection protocol; and
providing the patient population access to the data collection protocol.

14. A method for administering patient data, the method comprising:
establishing a survey protocol;
providing a patient access to the survey protocol;
collecting patient data inputs through the survey protocol;
aggregating patient data inputs collected from a plurality of patients; and
disseminating a summary of the aggregated patient data inputs.

15. The method of claim 14, wherein the survey protocol is a dynamic survey protocol.

16. The method of claim 15, wherein the dynamic survey protocol configures a current survey presented to a patient based on a previous survey presented to the patient.

17. The method of claim 14, further comprising:
determining a patient procedure for a patient population;
configuring, based on the patient procedure, the survey protocol for collecting patient inputs;
presenting the patient population with prompts; and
receiving patient data inputs based on the prompts.

18. The method of claim 14, further comprising:
establishing a first data set to disseminate to a first entity;
establishing a second data set to disseminate to a second entity;
disseminating the first data set to the first entity; and
disseminating the second data set to the second entity.

19. The method of claim 14, wherein disseminating the summary of the aggregated patient data inputs comprises disseminating a trend of the aggregated patient data.
